(19) United States
(12) Patent Application Publication
(10) Pub. No.: US 2024/0345651 A1
(43) Pub. Date: Oct. 17, 2024

(54) METHOD AND SYSTEM FOR DISPLAYING A VIRTUAL REALITY

(71) Applicant: DAYHOLI GMBH, Graz (AT)

(72) Inventor: Fjolla Holzleithner, Graz (AT)

(21) Appl. No.: 18/547,568

(22) PCT Filed: Feb. 25, 2022
(86) PCT No.: PCT/EP2022/054792

§ 371 Date: Aug. 23, 2023

(30) Foreign Application Priority Data
Feb. 25, 2021 (DE) 10 2021 104 553.3
Jun. 18, 2021 (DE) 10 2021 115 896.6
Jan. 4, 2022 (DE) 10 2022 100 108.3

(51) Int. Cl. G06F 3/01; A61M 21/00

(52) U.S. Cl. CPC G06F 3/011; A61M 21/00; A61M 2021/0016; A61M 2021/005

(57) ABSTRACT

A method and associated system display a visual virtual reality at a display location via a visual display unit. A generating device at the display location generates at least one surroundings property for the virtual reality. The surroundings property originates and is detected live at a recording location of the visual virtual reality and is received and generated live by the generating device at the display location.

The present invention relates to a method for displaying a virtual reality, in which a visual virtual reality is displayed at a display location by means of at least one visual display unit, and in which at least one surroundings property for the virtual reality is generated at the display location by means of at least one generating device.

### BACKGROUND

DE 10 2018 103 572 A1 describes a system for displaying a virtual reality.

The problem addressed by the present invention is therefore that of improving a display of the virtual reality.

### SUMMARY

The problem is solved by the subject matter having the features of the independent patent claims.

Additional objects and advantages of the invention will be set forth in part in the following description, or may be obvious from the description, or may be learned through practice of the invention.

The invention relates to a method for displaying a virtual reality. By means of the virtual reality, for example, locations can be displayed that cannot be easily reached. For example, natural places can be displayed that cannot be found at the display location. Alternatively, by means of the virtual reality, for example, a museum visit can also be displayed without the need to actually visit the museum. Therefore, remote locations, inaccessible locations or even fictitious locations can be made accessible by means of the virtual reality.

In the method, a visual virtual reality is displayed at a display location by means of at least one visual display unit. Images, for example, that correspond to the aforementioned museum or natural surroundings are displayed by means of the visual virtual reality. Additionally or alternatively, moving images can also be displayed by means of the visual virtual reality. The visual virtual reality corresponds to a visual display of the natural place or of the museum.

In addition, in the method, at least one surroundings property for the virtual reality is generated at the display location by means of at least one generating device. The virtual reality can be even more realistically displayed by means of the at least one surroundings property. In addition to the visual virtual reality, even more sensory impressions can therefore be generated for the virtual reality at the display location.

According to the invention, the at least one surroundings property, which originates from a recording location, is received live at the display location. The recording location is spatially separated from the display location. According to the aforementioned example, the recording location is, for example, a museum or the remote natural place. The display location can also be an apartment, so therefore spatially separated from the museum.

In addition, the at least one surroundings property is generated live at the display location by means of the generating device. Therefore, the surroundings property, which originates at the recording location, is received live and generated live at the display location, and so an even more realistic virtual reality can be displayed by means of this at least one surroundings property. The surroundings property, which is at least received and generated live, can also be referred to as a live surroundings property. A user therefore receives the at least one surroundings property that prevails at the recording location at the moment live at the display location, i.e., for example, in his or her apartment. The virtual reality therefore becomes even more realistic. Not only is the visual virtual reality displayed at the display location, i.e., for example, the visual surroundings in the museum with the paintings or statues, but the live surroundings properties are displayed as well.

It is advantageous when visual data from the recording location for displaying the visual virtual reality are received live at the display location and are displayed live at the display location by means of the visual display unit. Additionally or alternatively, audio data from the recording location for representing sounds and/or language at the display location can be received live at the display location and played live at the display location by means of at least one loudspeaker. Additionally or alternatively, acoustic data from the recording location for representing an acoustic virtual reality can also be received live at the display location and played live at the display location by means of an acoustic playback unit, for example, a loudspeaker.

It is advantageous when the at least one surroundings property can be transmitted live from the recording location to the display location. The at least one surroundings property is therefore transmitted live, received live at the display location and generated live at the display location. Additionally, the visual data for displaying the visual virtual reality can also be transmitted live, received live at the display location and displayed or generated live at the display location.

It is advantageous when the at least one surroundings property is detected at the recording location in real time, transmitted from the recording location to the display location in real time, received at the display location in real time and/or generated at the display location in real time.

It is advantageous when wind, a wind direction, a fragrance, temperature and/or atmospheric humidity are/is detected as a surroundings property at the recording location. Additionally or alternatively, it is advantageous when wind, fragrance, temperature and/or atmospheric humidity according to the surroundings property detected at the recording location are/is generated at the display location. Furthermore, additionally or alternatively, brightness and/or light conditions can also be detected and/or generated at the display location. As a result, the virtual reality can be more realistically displayed. The user therefore not only sees the surroundings or the recording location or a display of the recording location, but can also feel the recording location, for example, wind conditions and/or a fragrance.

It is advantageous when the at least one surroundings property is detected at the recording location by means of at least one surroundings sensor. The surroundings sensor can be, for example, a wind sensor, a wind direction sensor, a temperature sensor, a fragrance sensor, an atmospheric humidity sensor, a brightness sensor and/or a light sensor.

It is advantageous when the virtual reality is displayed by means of a control unit, wherein the control unit preferably receives the at least one surroundings property by means of a data interface. In particular, the at least one surroundings property is received live by means of the data interface. The data interface can therefore at least receive data.

It is advantageous when the detection of the at least one surroundings property at the recording location is carried out according to instructions from the display location. For example, the data interface can also be designed to send data, i.e., for example, the instructions. Data can be bidirectionally transmitted by means of the data interface. For example, a user at the display location can transmit instructions to the recording location to change a viewing direction so that the user sees another area of the museum at the display location.

It is advantageous when the control unit requests the at least one surroundings property from the recording location. Additionally or alternatively, the detection of the at least one surroundings property at the recording location can be controlled and/or carried out according to instructions, in particular, by means of the control unit. For example, the user can give instructions at the recording location, from the display location, for a viewing direction to be changed and/or to move in the natural place in a direction of his/her choosing. During the movement in the natural place, the surroundings property can also change, of course, wherein the surroundings property that suits the movement or that changes with the movement can be displayed at the display location.

It is advantageous when the control unit ascertains the at least one surroundings property at the recording location by means of the at least one surroundings sensor. For example, the control unit can also ascertain a brightness at the recording location by means of a camera as a surroundings sensor, by means of which the visual data for displaying the visual virtual reality are gathered.

It is advantageous when the generating device includes at least one fan, by means of which the wind and/or the wind direction are/is generated. Additionally or alternatively, the generating device can include at least one nozzle, by means of which an odorant is dispensed, which generates the fragrance. The odorant can also be dispensed in the area of the fan so that the odorant reaches the user. In addition, a location at which the fragrance arises or a direction from which the fragrance comes can therefore also be simulated. It is advantageous when multiple fans and/or nozzles are present so that a direction of the wind and/or of the fragrance can be represented. The at least one fan and/or the at least one nozzle can be regulated with respect to intensity and/or can be swiveled. A wind force can be represented as a surroundings property, in particular by controlling the intensity of the at least one fan. The generating device that generates the fragrance can also be referred to as a fragrance device or can be a generating device.

It is advantageous when the generating device includes a fragrance device, in which the odorant or the fragrance is preferably mixed from multiple basic substances. As a result, a plurality of fragrances can be generated. As described above, the generating device can also be the fragrance device. Additionally or alternatively, the generating device or the fragrance device can also include the odorants, from which the fragrance is directly formed.

The invention further relates to a system for displaying a virtual reality. Moreover, the system can be operated according to at least one method step of the preceding description and/or of the following description. The system can also be designed such that it can carry out the method according to at least one method step of the preceding description and/or of the following description.

The system includes at least one visual display unit for displaying a visual virtual reality at a display location. The virtual reality can be, for example, a display of a museum or of a natural place, which are displayed in a private apartment as the display location. The advantages are that, by means of the virtual reality, remote locations, inaccessible locations or even fictitious locations can be visited without the need to leave one's apartment.

The system also includes at least one generating device for generating at least one surroundings property for the virtual reality at the display location. The virtual reality can be even more realistically displayed by means of the at least one surroundings property.

Furthermore, the at least one surroundings property, which originates from a recording location, can be received live at the display location. Moreover, the at least one surroundings property can be generated live by means of the at least one generating device. The surroundings property can therefore be received and generated live at the display location. As a result, the virtual reality can be more realistically represented.

It is advantageous when the system includes a control unit, by means of which the virtual reality can be displayed. The visual virtual reality can be displayed by means of the control unit and the surroundings properties can be generated by means of the generating device.

It is advantageous when the control unit can receive the at least one surroundings property live by means of a data interface and generate the at least one surroundings property live by means of the at least one generating device.

It is advantageous when the control unit can control the detection of the at least one surroundings property at the receiving location, in particular by means of the data interface. Additionally or alternatively, it is advantageous when the control unit can request the at least one surroundings property from the recording location, in particular by means of the data interface. As a result, the virtual reality can be controlled from the display location. The control unit is preferably arranged at the display location.

It is advantageous when, by means of the control unit, the at least one surroundings property can be ascertained at the recording location by means of at least one surroundings sensor. The control unit can be designed such that said control unit can ascertain the at least one surroundings property at the recording location by means of the surroundings sensor. For example, the control unit can ascertain brightness or light conditions by means of a camera as a surroundings sensor. Additionally or alternatively, the control unit can also ascertain, in addition to the wind force, a wind direction by means of a wind sensor as a surroundings sensor.

It is advantageous when the control unit can establish a connection to at least one surroundings sensor at the recording location by means of the data interface in order to be able to receive the at least one surroundings property live and/or ascertain the at least one surroundings property.

It is advantageous when the generating device includes at least one fan and/or at least one fragrance device having at least one nozzle. The wind, for example, as a surroundings property, can be generated by means of the at least one fan. The intensity of at least one fan can also be regulated so that a wind force, as a surroundings property, can also be generated. The generating device can also be the fragrance device. The generating device or the fragrance device can also include the containers for the basic substances and/or containers that contain the odorants. It is also advantageous when the containers are exchangeable so that empty containers can be exchanged for new containers.

It is advantageous when the fragrance device includes multiple containers for storing basic substances, wherein the fragrance device can mix the fragrances by means of the basic substances. As a result, a plurality of fragrances can be mixed from a small number of various basic substances. Additionally or alternatively, the generating device or the fragrance device can also include the odorants, from which the fragrance is directly formed. The generating device or the fragrance device can also include the containers for the basic substances and/or containers that contain the odorants. It is also advantageous when the containers are exchangeable so that empty containers can be exchanged for new containers.

The system can also include a visual detection unit, by means of which the visual data for displaying the visual virtual reality can be gathered at the recording location. Additionally or alternatively, the system can also include at least one microphone in order to be able to record sound at the recording location.

The invention also relates to a fragrance device for generating fragrances for the virtual reality. The fragrance device can be provided and/or designed for a system according to at least one feature of the preceding description and/or the following description. Additionally or alternatively, the fragrance device can be provided and/or designed for a method according to at least one feature of the preceding description and/or of the following description. Additionally or alternatively, the fragrance device can also have at least one feature according to the preceding description and/or the following description. Additionally or alternatively, the fragrance device can also have at least one feature according to the preceding description and/or the following description, which are described in conjunction with the generating device.

The fragrance device includes at least one container for storing at least one odorant. Additionally or alternatively, the fragrance device can also include at least one container, in which a basic substance is contained, by means of which the odorant can be formed, in particular mixed.

Moreover, the fragrance device includes at least one distributing unit for distributing the odorant at the display location.

Furthermore, the fragrance device includes at least one control unit for controlling the fragrance device so that fragrances corresponding to the virtual reality can be formed.

It is advantageous when the fragrance device includes multiple containers for storing basic substances and/or odorants. Moreover, the fragrance device can mix the fragrances by means of the basic substances. The fragrance device can include a mixing device for this purpose.

It is advantageous when the containers are exchangeable. As a result, full containers can be installed when the containers are empty.

It is advantageous when the fragrance device includes at least one fan and/or at least one nozzle as the distributing unit. The odorant and/or the basic substances that can form the odorants, for example, in the air, can be dispensed by means of the nozzle. The nozzle can preferably be associated with the fan so that the fan distributes the fragrances at the display location and conveys the odorants to the user.

In addition, the fragrance device can include multiple fans and/or multiple nozzles. As a result, a spatial olfactory experience can be represented or multiple fragrances can be distributed. Furthermore, one nozzle can be associated with each fan.

It is advantageous when the control unit is designed such that scented surroundings can be created by means of the control unit. Various scented surroundings or fragrance scenarios can be stored in the control unit or in a memory connected to the control unit and can be selected. For example, a field of flowers can be stored as the scented surroundings or the fragrance scenarios so that smelling a field of flowers can be experienced in the virtual reality.

Additionally or alternatively, the control unit can also include a data interface, by means of which the surroundings properties can be received, in particular live, from the recording location. As a result, the fragrances at the display location can be adapted to the fragrances at the recording location. It can also suffice when only position data are transmitted from the recording location to the control unit. The control unit can then, in particular automatically, ascertain which fragrances are present at the recording location and generate the corresponding fragrances at the display location. For example, the control unit ascertains that the recording location is on a field of flowers and can then generate appropriate fragrances at the display location.

### BRIEF DESCRIPTION OF THE DRAWINGS

Further advantages of the invention are described in the following exemplary embodiments, wherein:

FIG. 1 shows a schematic view of a system for displaying a virtual reality, and

FIG. 2 shows a schematic view of a fragrance device.

### DETAILED DESCRIPTION

In the following description of the alternative exemplary embodiments represented in the figures, the same reference signs are utilized for features that are identical or at least comparable in terms of their configuration and/or mode of operation. Provided the features are not described in detail again, their design and/or mode of operation correspond/corresponds to the design and mode of operation of the above-described features. For the sake of greater clarity, reference signs for previously described components have not been individually included in the figures.

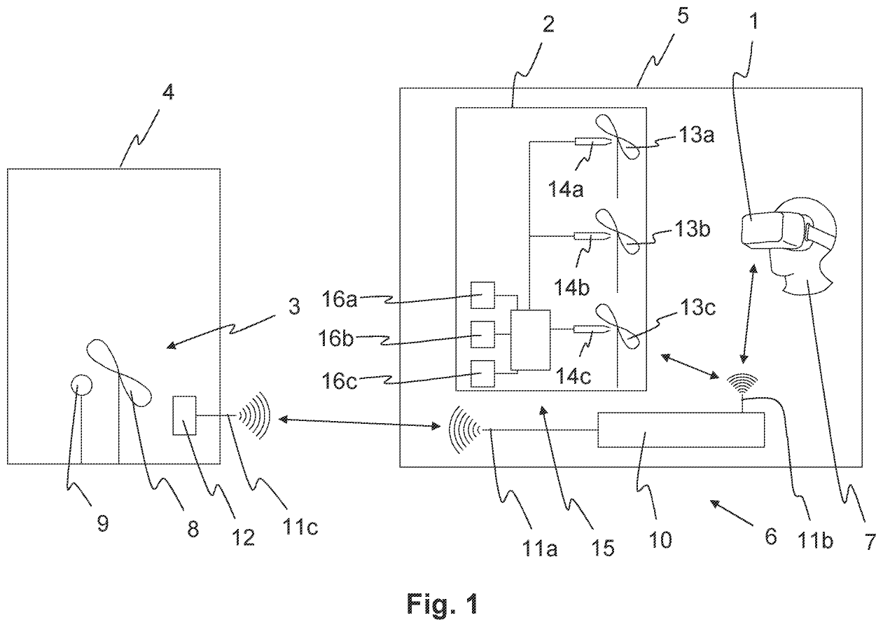

FIG. 1 shows a schematic view of a system 6 for displaying a virtual reality.

The system 6 includes at least one virtual display unit 1, by means of which a visual virtual reality is displayed. In this exemplary embodiment, the visual display unit 1 is a pair of goggles, so-called VR glasses (virtual reality glasses), which are worn by a user 7. Alternatively, the visual display unit 1 can also be a monitor, a screen or a projector. A visual virtual reality can be displayed by means of the visual display unit 1. This visual virtual reality can include images or videos. The visual virtual reality can be displayed at a display location 5 by means of the visual display unit 1. The display location 5 is the location at which the user 7 is located. For example, the display location 5 can be an apartment where the virtual reality is displayed. The advantage of the virtual reality is that, for example, a museum can be visited using the virtual reality, without the need to visit the museum. The museum visit described here can therefore conveniently take place at home. As another example, natural places that are remote, inaccessible or purely fictitious can also be visited using the virtual reality. For example, a sea or an ocean can also be visited by means of the virtual reality. Additionally, sounds and/or language that suit the virtual reality can also be generated.

Moreover, the system 6 includes at least one generating device 2 for generating at least one surroundings property for the virtual reality at the display location 5. The surroundings property or the surroundings properties are, for example, wind conditions, such as wind direction and/or wind force, fragrances, temperature, atmospheric humidity, brightness and/or light conditions. Generating the at least one surroundings property allows the virtual reality to be even more realistically displayed. For example, not only can the visit to an ocean therefore be displayed visually by means of the visual display unit 1, but rather at least one surroundings property can also be generated. At the ocean, therefore, not only can the view of the ocean be displayed, but rather an onshore or offshore wind and/or an appropriate salty smell can also be represented.

In the method for displaying the virtual reality, the at least one surroundings property, which originates from a recording location 4, is received live at the display location 5. Moreover, in the method, the at least one surroundings property is generated live at the display location 5 by means of the generating device 2. The surroundings property, which originates from the recording location 4, is therefore received and generated live at the display location 5. Therefore, the at least one surroundings property that prevails at the recording location 4 at the moment is displayed at the display location 5 by means of the virtual reality. A live surroundings property is therefore displayed at the display location 5. The surroundings property is therefore received and generated in real time at the display location 5. Furthermore, the live surroundings properties suit the visual data that are currently displayed and/or the sounds that are represented. If trees are visually displayed that are moving, for example, due to wind, the corresponding wind is also generated as a surroundings property. The visual data, the sounds or the noise and the at least one generated surroundings property all suit one another. The corresponding visual virtual reality, the acoustic virtual reality and the virtual reality corresponding to the surroundings property therefore also suit one another.

The at least one surroundings property can also be transmitted from the recording location 4 to the display location 5 live and/or in real time.

According to the present exemplary embodiment, at least one surroundings sensor 3 is arranged at the recording location 4, as is schematically represented. The at least one surroundings property can be detected at the recording location 4 by means of the at least one surroundings sensor 3. Additionally or alternatively, the at least one surroundings property can be ascertained at the recording location 4 by means of the at least one surroundings sensor 3. By way of example, a wind sensor 8 and a fragrance sensor 9 are shown here as surroundings sensors 3. Multiple appropriate sensors 3 can also be used and provided. Additionally or alternatively, a temperature sensor, a light sensor, a brightness sensor and/or a humidity sensor can also be provided.

Moreover, the system 6 according to the present exemplary embodiment also includes a control unit 10, by means of which the display of the virtual reality is controlled or can be controlled.

The control unit 10 according to the present exemplary embodiment also includes at least one data interface 11a, 11b. The at least one surroundings property can be received live by means of the at least one data interface 11a, 11b. The surroundings property can then be generated live. The control unit 10 in this case has a first data interface 11a in order to be able to receive the surroundings property live, in particular from the recording location 4.

By means of the first data interface 11a, the surroundings properties can be received live and/or a connection to the recording location 4 can be established in order to be able to receive the surroundings property. Additionally or alternatively, the visual data and/or the acoustic data for displaying the visual virtual and/or acoustic reality can also be received, preferably live and in real time, by means of the first data interface 11a. These visual and/or acoustic data can also be displayed live and in real time by means of the visual display unit 1.

A communication unit 12 can be arranged at the recording location 4 in order to be able to transmit the surroundings property to the display location 5. As shown here, the communication unit 12 has a third data interface 11c. The communication unit 12 can also be designed to gather and/or evaluate the surroundings property detected by the surroundings sensor 3. The communication interface 12 can also transmit the surroundings property to the display location 5, in particular live and in real time, by means of the third data interface 11c. At the display location 5, the first data interface 11a can receive the surroundings property live and in real time. A connection between the recording location 4 and the display location 5, or between the third data interface 11c and the first data interface 11a, which is represented here merely by means of an arrow, can be, for example, the Internet. The visual data can also be transmitted, in particular live and in real time, via the same connection.

The control unit 10 shown here includes, according to the present exemplary embodiment, a second data interface 11b, by means of which a connection can be established with the visual display unit 1 and the generating device 2, the connection being schematically represented by means of arrows. Alternatively, the control unit 10 can also have a data interface 11 for the visual display unit 1 and a data interface 11 for the generating device 2.

The at least one surroundings property is therefore transmitted to the at least one generating device 2 for the live and real-time generation of the surroundings property.

The control unit 10 can also control and/or request, in particular by means of the first data interface 11a, the gathering of the visual data and/or the acoustic data and/or the at least one surroundings property. For example, a person at the recording location 4 can also be instructed to turn in one direction or to move in one direction. For example, a drone can also be arranged at the recording location 4, which can gather the visual data and/or the acoustic data and/or the at least one surroundings property. The drone can be moved by means of the control unit 10. In addition or as an alternative to the drone, a controllable vehicle or the like can also be arranged at the recording location 4.

According to the present exemplary embodiment, the generating device 2 includes at least one fan 13a through 13c. Wind can therefore be generated as a surroundings property by means of the at least one fan 13a through 13c. The at least one fan 13a through 13c can be controllable in order to be able to represent a wind force. Additionally or alternatively, the at least one fan 13a through 13c can also be swivelable in order to be able to represent a wind direction. According to the present exemplary embodiment, multiple, namely three in this case, fans 13a through 13c are shown, and so, as a result, the wind can be presented from multiple directions. The three fans 13a through 13c are situated in a special arrangement with respect to the user 7.

Moreover, the generating device 2 according to the present exemplary embodiment includes at least one nozzle 14a through 14c, by means of which odorants can be distributed at the display location 5 in order to be able to present a fragrance as a surroundings property. The at least one nozzle 14a through 14c can be arranged in the area of the at least one fan 13a through 13c in order to convey the odorant to the user 7. According to the present exemplary embodiment, multiple, namely three in this case, nozzles 14a through 14c are shown. As a result, a directionality of the fragrance can be achieved. The user 7 can therefore be shown that a fragrance comes from a certain direction. Moreover, one nozzle 14a through 14c is associated with each of the fans 13a through 13c, respectively. Of course, a nozzle 14a through 14c can also be associated with only a portion of the fans 13a through 13c.

The generating device 2 also includes a fragrance device 15 for forming the fragrances. The fragrance device 15 is connected to the at least one nozzle 14a through 14c, for example, by means of lines, in order to be able to conduct the odorant to the nozzle 14a through 14c.

The fragrance device 15 also includes multiple containers 16a through 16c and/or is coupled thereto, wherein basic substances for the fragrances are arranged in each of the containers 16a through 16c. The fragrance device 15 can mix the odorants by means of the multiple basic substances so that multiple different fragrances can be formed.

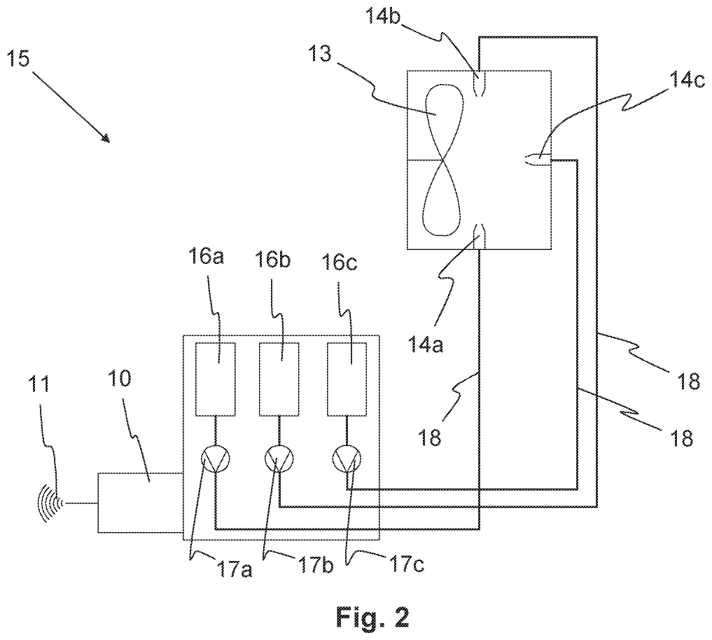

FIG. 2 shows a schematic view of the fragrance device 15 for generating fragrances at the display location 5. The fragrance device 15 shown here can also be the generating device 2 or an embodiment of the generating device 2. Alternatively, the generating device 2 can include the fragrance device 15. Alternatively, the fragrance device 15 can also include the generating device 2.

Features that have already been described with reference to the at least one preceding figure are not explained once more, for the sake of simplicity. Moreover, features can also be described first in this figure or in at least one of the following figures. Moreover, identical reference characters are utilized for identical features for the sake of simplicity. In addition, for the sake of clarity, not all the features may be shown and/or labeled with a reference character in the following figures. Features shown in one or several of the preceding figures can also be present in this figure or in one or several of the following figures, however. Moreover, for the sake of clarity, features can also be shown and/or labeled with a reference character first in this figure or in one or several of the following figures. Nevertheless, features that are first shown in one or several of the following figures can also be already present in a preceding figure.

The fragrance device 15 includes at least one container 16a through 16c for storing odorants and/or the basic substances. The containers 16a through 16c can be exchangeable in order to be able to exchange empty containers for new containers 16a through 16c or in order to supply the fragrance device 15 with new fragrances.

Moreover, the fragrance device 15 includes at least one distributing unit 13, 14, which in this case is a fan 13 and/or a nozzle 14. The fragrance device 15 can also include multiple fans 13 and/or nozzles 14. Moreover, at least one nozzle 14 can be associated with each fan 13 in order to dispense the odorant when generating the wind for the virtual reality and in order to convey the odorant by means of the wind.

According to the present exemplary embodiment, three nozzles 14a through 14c are associated with the fan 13 in order to be able to dispense various odorants. Moreover, according to the present exemplary embodiment, each nozzle 14a through 14c is connected by means of lines 18 to a container 16a through 16c, respectively. The fragrance device 15 includes at least one pump 17a through 17c in order to convey the odorants to the nozzles 14a through 14c. One pump 17a through 17c is associated with each container 16a through 16c, respectively, and/or each nozzle 14a through 14c, respectively. As a result, mixing the odorants from the containers 16a through 16c can be avoided when, for example, there is an odorant in the containers 16a through 16c. However, when there are basic substances for the odorants in the containers 16a through 16c, it can be advantageous when the fragrance device 15 includes a mixing device in order to appropriately mix the basic substances. In this case, the pumps 17a through 17c can pump the basic substances out of the containers 16a through 16c into the mixing device. Alternatively, the basic substances can also be conducted to the fan 13, wherein the basic substances become mixed in the air flow there to form the odorant.

According to the present exemplary embodiment, the control unit 10 is associated with the fragrance device 15. This can be the control unit 10 from FIG. 1 or an additional control unit 10, which is associated with the fragrance device 15 or which includes the fragrance device 15.

The fragrance device 15 or the control unit 10 has a data interface 11 in order to be able to receive, in particular live, data from the recording location 4. By means of these data from the recording location 4, a fragrance that suits the recording location 4 can be subsequently generated for the virtual reality by means of the fragrance device 15.

The control unit 10 shown here, as well as the control unit 10 from FIG. 1, can also include a memory (not shown here) in order to store fragrance information. The fragrances that suit the recording location 4 can be generated by means of the memory and the fragrance information. For example, data regarding the fragrances can be transmitted to the control unit 10 and the fragrances are then generated. Additionally or alternatively, data regarding the recording location 4 can also be transmitted, on the basis of which appropriate fragrances are then generated. For example, data are transmitted that indicate that a field of flowers is present at the recording location 4. An appropriate fragrance can then be generated on the basis of these data. Additionally or alternatively, GPS data can also be transmitted. On the basis of the GPS data, the control unit 10 can then ascertain which fragrances are present at the recording location 4, whereupon the fragrances are then generated.

The present invention is not limited to the represented and described exemplary embodiments. Modifications within the scope of the claims are also possible, as is any combination of the features, even if they are represented and described in different exemplary embodiments.

### LIST OF REFERENCE CHARACTERS

1 visual display unit
2 generating device
3 surroundings sensor
4 recording location
5 display location
6 system
7 user
8 wind sensor
9 fragrance sensor
10 control unit
11 data interface
12 communication unit
13 fan
14 nozzle
15 fragrance device
16 container
17 pump
18 line

1-23: (canceled)

24. A method for displaying a virtual reality, comprising:
displaying a visual virtual reality at a display location via a visual display unit;
with a generating device at the display location, generating at least one surroundings property for the virtual reality; and
wherein the surroundings property originates and is detected live at a recording location of the visual virtual reality and is received and generated live by the generating device at the display location.

25. The method of claim 24, comprising transmitting visual data live from the recording location and displaying the visual data live for the visual virtual reality at the display location via the visual display unit.

26. The method of claim 24, wherein the surroundings property is transmitted live from the recording location to the display location.

27. The method of claim 24, wherein the surroundings property is detected at the recording location in real time, transmitted from the recording location to the display location in real time, received at the display location in real time, and generated at the display location in real time.

28. The method of claim 24, wherein the surroundings property detected at the recording location and generated at the display location is one or more of: wind, a wind direction, a fragrance, temperature, or atmospheric humidity.

29. The method of claim 24, wherein the surroundings property is detected at the recording location by a surroundings sensor.

30. The method of claim 24, wherein the virtual reality is displayed by a control unit at the display location, the control unit receiving the surroundings property via a data interface with the recording location.

31. The method of claim 30, wherein the detection of the surroundings property at the recording location is carried out according to instructions from the control unit at the display location.

32. The method of claim 31, wherein the control unit requests the surroundings property from the recording location.

33. The method of claim 32, wherein the control unit ascertains the surroundings property at the recording location via a surroundings sensor at the recording location.

34. The method of claim 24, wherein the generating device includes one or more of:
a fan to generate wind or wind direction; or
a nozzle to create a fragrance by dispensing an odorant from an odorant source, wherein the fan disperses the odorant.

35. The method of claim 34, wherein the generating device includes a fragrance device in which the odorant is mixed from multiple basic substances.

36. A system for displaying a virtual reality, comprising:
a visual display unit to display a visual virtual reality at a display location;
a generating device configured to generate a surroundings property for the virtual reality at the display location; and
wherein the surroundings property is detected at a recording location of the virtual reality, transmitted to the display location, and generated live at the display location by the generating device.

37. The system of claim 36, further comprising a control unit at the display location that controls display of the virtual reality.

38. The system of claim 37, wherein the control unit receives the surrounding property live from the recording location via a data interface.

39. The system of claim 38, wherein the control unit is configured to request or control detection of surroundings property at the recording location via the data interface.

40. The system of claim 39, wherein the control unit is in communication with a sensor at the recording location that ascertains the surroundings property.

41. The system of claim 40, wherein the control unit is in communication with the sensor via the data interface.

42. The system of claim 36, wherein the generating device comprises one or more of:
a fan to generate wind or wind direction; or
a fragrance device that includes a nozzle to create a fragrance by dispensing an odorant from an odorant source, wherein the fan disperses the odorant.

43. The system of claim 42, wherein the fragrance device comprises multiple containers that store different odorants or basic substances that are mixed to create different fragrances.

44. A fragrance device for generating fragrances in a virtual reality system, comprising:
a container that stores an odorant or basic substance used in mixing an odorant;
a distributing unit that disperses the odorant at a display location where the virtual reality is presented; and
a control unit configured to control the distributing unit to disperse the odorant at a time coordinated with a visual display of the virtual reality.

45. The fragrance device of claim 44, comprising a plurality of the containers, each container having a different odorant or a different basis substance for mixing different odorants.

46. The fragrance device of claim 44, further comprising a fan and a nozzle for dispersing the odorant from the containers.
